(19) United States
(12) Patent Application Publication
(10) Pub. No.: US 2014/0193810 A1
(43) Pub. Date: Jul. 10, 2014

(54) LIQUID INJECTION JIG SET

(71) Applicant: Sony Corporation, Tokyo (JP)

(72) Inventor: Toshio Watanabe, Kanagawa (JP)

(73) Assignee: SONY CORPORATION, Tokyo (JP)

(21) Appl. No.: 14/084,996

(22) Filed: Nov. 20, 2013

(30) Foreign Application Priority Data
Jan. 4, 2013 (JP) 2013-000225

(51) Int. Cl. C12Q 1/68

(52) U.S. Cl. CPC C12Q 1/6806
USPC 435/6.11; 141/329

(57) ABSTRACT

A liquid injection jig set includes a chip receiver in which a microchip having a plate shape is received in a case, and a jig that is configured to be fittable to the chip receiver includes a hollow needle that can pierce a side end surface of the microchip, which is exposed from the case, and introduces liquid into the microchip via the hollow needle.

### CROSS REFERENCE TO RELATED APPLICATIONS

This application claims the benefit of Japanese Priority Patent Application JP 2013-000225 filed Jan. 4, 2013, the entire contents of which are incorporated herein by reference.

### BACKGROUND

The present disclosure relates to a liquid injection jig set. More specifically, the present disclosure relates to the liquid injection jig set that includes a chip receiver constituted by a microchip and a case, and a jig and that is used for injecting liquid into the microchip.

In recent years, a microchip in which a well or a passage is formed on a silicon or glass-based substrate to conduct a chemical and biological analysis has been developed by applying a piece of micro-machining technology in the semiconductor industry. Such a microchip has been used for an electrochemical detector of liquid chromatography, a compact electrochemical sensor in the medical field, or the like, for example.

An analysis system using such a microchip is called a micro-Total-Analysis System (a μ-TAS), a lab-on-a-chip, a bio chip, or the like and attracts attention as a piece of technology enabling high speed, high efficiency of a chemical and biological analysis, integration of the device, or a compact analysis device. The μ-TAS enables an analysis with a small amount of samples or a disposable use of a microchip. Thus application, particularly, to a biological analysis which deals with a trace amount of a valuable sample or a large number of specimens is expected for the μ-TAS.

An optical detection device in which a substance is introduced into a plurality of areas on a microchip and the substance is chemically detected is exemplified as an application example of the μ-TAS. A reactor (a real-time PCR device, for example) in which a reaction, such as a nucleic acid amplification reaction, among a plurality of substances is progressed in a well on a microchip and the substance generated is optically detected is exemplified as an example of such an optical detection device.

The above-described analysis using the μ-TAS is conducted with a trace amount of a sample. Thus, in some cases, it is difficult to introduce the sample into an area, such as a well, provided on a microchip.

To solve the problem described above, “a sample liquid supply jig including a receiving trunk potion into which liquid is injected, a hollow needle which is provided on one end of the receiving trunk portion and of which the hollow portion is in communication with an inner portion of the receiving trunk portion, and a seal portion which covers a part of the receiving trunk portion, into which the liquid is injected, and has a self-sealing property by elastic deformation” has been disclosed in Japanese Unexamined Patent Application Publication No. 2012-159337. In the sample liquid supply jig, the microchip is pierced, at a predetermined position, by the hollow needle which is in communication with the inner portion of the receiving trunk portion capable of holding the sample liquid. Therefore, the receiving trunk portion is connected inside the microchip, and thus it is possible to supply the sample liquid.

### SUMMARY

According to the above-described sample liquid supply jig provided with the hollow needle, it is possible to easily introduce a trace amount of the sample liquid into the microchip. However, to accurately introduce the sample into the microchip using the hollow needle, it is necessary for the hollow needle to pierce an appropriate portion of the microchip which is provided with a fine structure, such as a well or a passage. Here, it is desirable to provide a liquid injection jig set which enables introduction of liquid into a microchip with high accuracy.

That is, according to an embodiment of the present disclosure, there is provided a liquid injection jig set including a chip receiver in which a microchip having a plate shape is received in a case, and a jig that is configured to be fittable to the chip receiver includes a hollow needle that can pierce a side end surface of the microchip, which is exposed from the case, and introduces liquid into the microchip via the hollow needle.

It is preferable that the chip receiver be positioned to and fitted to the jig, and it is more preferable that the chip receiver be positioned in a right-left direction and an up-down direction.

In addition, the liquid injection jig set may include a structure that regulates a fitting direction of the chip receiver to the jig.

The jig may have a structure enabling pinching of the chip receiver. Furthermore, the jig may include two plate portions opposite to each other and the chip receiver may be fitted to a gap between the two plate portions.

The respective plate portions may be different in length and/or width. A reaction field formed in the microchip may be visually confirmed through at least either one of the two plate portions, in a state where the chip receiver is fitted to the jig.

It is preferable that the liquid be subjected to negative pressure suction into the microchip when the hollow needle pierces the microchip.

In addition it is preferable that the microchip be configured to have a substrate layer including a polydimethylsiloxane layer and the hollow needle pierce the polydimethylsiloxane layer.

According to another embodiment of the present disclosure, there is provided a chip receiver including a microchip having a plate shape, and a case capable of receiving the microchip, in which the case is configured to be fittable to a jig that has a hollow needle that can pierce a side end surface of the microchip and introduces liquid into the microchip via the hollow needle, and the side end surface of the microchip is exposed in a state where the microchip is inserted in the case.

The chip receiver may include a structure that regulates an insertion direction of the microchip into the case.

In addition, the chip receiver may include a locking portion that restricts movement of the microchip received in the case.

According to still another embodiment of the present disclosure, there is provided a jig including a structure which enables fitting of a chip receiver in which a microchip having a plate shape is received in a case, and a hollow needle that can pierce a side end surface of the microchip, which is exposed from the chip receiver, in which liquid is introduced into the microchip via the hollow needle.

The present disclosure is to provide a liquid injection jig set or the like which enables introduction of liquid into a microchip with high accuracy.

### BRIEF DESCRIPTION OF THE DRAWINGS

FIGS. 1A and 1B are views explaining a configuration of a liquid injection jig set according to a first embodiment of the present disclosure;

FIGS. 2A to 2C are views explaining a configuration of a microchip which constitutes the liquid injection jig set according to the first embodiment of the present disclosure;

FIGS. 3A to 3C are views explaining a configuration of a case which constitutes the liquid injection jig set according to the first embodiment of the present disclosure;

FIGS. 4A to 4C are views explaining a configuration of a jig which constitutes the liquid injection jig set according to the first embodiment of the present disclosure;

FIGS. 5A to 5C are views explaining introduction of liquid into the microchip in the liquid injection jig set according to the first embodiment of the present disclosure;

FIG. 6 is a view explaining a configuration of a liquid injection jig set according to a modification embodiment of the first embodiment; and

FIGS. 7A and 7B are views explaining a configuration of a liquid injection jig set according to a second embodiment of the present disclosure.

### DETAILED DESCRIPTION OF EMBODIMENTS

Hereinafter, a preferred embodiment for implementing the present disclosure will be described. In addition, embodiments described below are intended only to show an exemplary embodiment of the present disclosure, and the scope of the present disclosure is not interpreted to be limited by these embodiments. The description will be given in the following order.

1. Configuration of Liquid Injection Jig Set according to First Embodiment of Present Disclosure
(1) Chip Receiver
(1-1) Microchip
(1-2) Case
(2) Jig
(2-1) Plate Portion
(2-2) Hollow Needle
(2-3) Connection Portion
   2. Injection of Liquid by Liquid Injection Jig Set according to First Embodiment of Present Disclosure
   3. Configuration of Liquid Injection Jig Set according to Modification Embodiment of First Embodiment
   4. Configuration of Liquid Injection Jig Set according to Second Embodiment of Present Disclosure
(1) Connection Portion
(2) Plate Portion

### 1. Configuration of Liquid Injection Jig Set According to First Embodiment of Present Disclosure

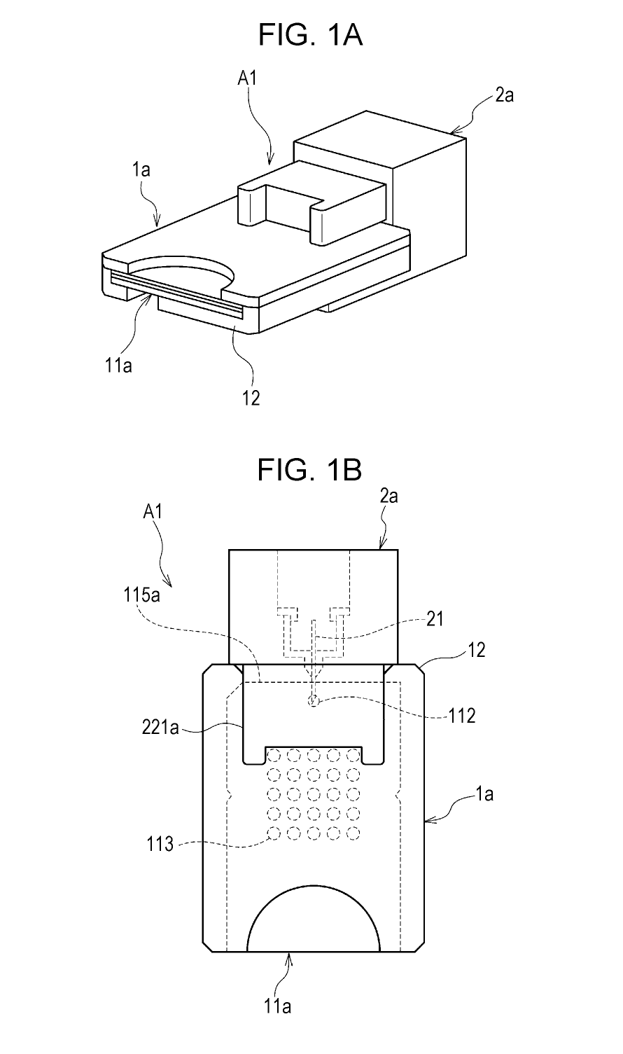

FIGS. 1A and 1B are schematic views of a liquid injection jig set A1 according to a first embodiment of the present disclosure. FIG. 1A is a perspective view of an external appearance of the liquid injection jig set A1, and FIG. 1B is a top view of the liquid injection jig set A1. This liquid injection jig set A1 includes a chip receiver 1a in which a microchip 11a having a plate shape is received in a case 12, and a jig 2a that is configured to be fittable to the chip receiver 1a includes a hollow needle 21 that can pierce a side end surface 115a of the microchip 11a, which is exposed from the case 12, and introduces liquid into the microchip 11a via the hollow needle 21. Hereinafter, details of each configuration of the liquid injection jig set A1 according to the first embodiment will be described.

### (1) Chip Receiver

It is roughly said that the chip receiver 1a has the microchip 11a and the case 12. Details of each configuration of these microchip 11a and case 12 will be described in order.

### (1-1) Microchip

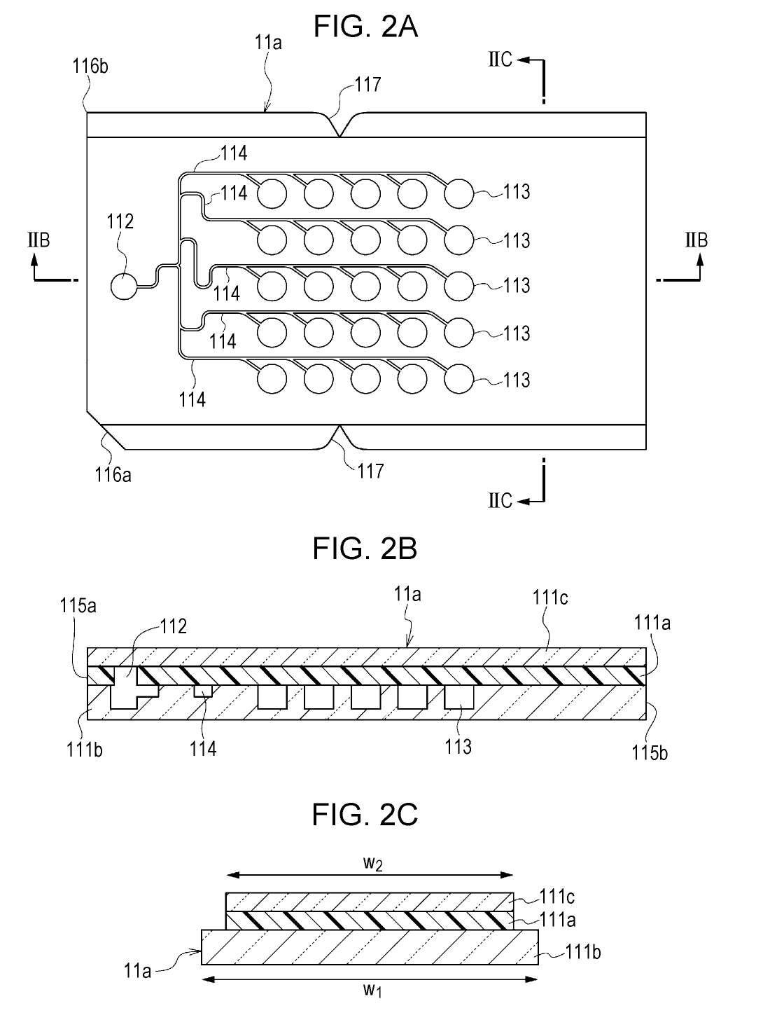

FIGS. 2A to 2C are schematic views of the microchip 11a. FIG. 2A is a top view of the microchip 11a, FIG. 2B is a cross-sectional view of the microchip 11a taken along line IIB-IIB indicated by arrows in FIG. 2A, and FIG. 2C is a cross-sectional view of the microchip 11a taken along line IIC-IIC indicated by arrows in FIG. 2A. As shown in FIG. 2A, the microchip 11a has an introduction portion 112 into which liquid is introduced from an outside. Furthermore, the microchip 11a is provided with a well 113 as a reaction field in which reaction is conducted for a chemical analysis or a biological analysis. These introduction portion 112 and well 113 are connected to each other via a passage 114. In addition, the figure of the microchip 11a is not limited to the figure shown in FIGS. 2A to 2C.

The liquid introduced into the microchip 11a is liquid-state substance containing an analyte or a substance which reacts with other substances and produces an analyte. Examples of the analyte include nucleic acids such as DNA or RNA, proteins which contain peptides, antibodies or the like, and the like. In addition, a biological sample itself containing the analyte described above, such as blood, swabs (nasal or throat swabs, nasal mucus, phlegm or the like), tears, urine, or the diluted solution thereof may also be used as the liquid introduced into the microchip 11a.

Examples of an analysis method using the microchip 11a include analysis methods using a nucleic acid amplification reaction, such as the known Polymerase Chain Reaction (PCR) method in which a temperature cycle is carried out, or various isothermal amplification methods in which a temperature cycle is not performed. Examples of the isothermal amplification method include a Loop-Mediated Isothermal Amplification (LAMP) method, a Smart Amplification Process (SMAP) method, a Nucleic Acid Sequence-Based Amplification (NASBA) method, an Isothermal and Chimeric primer-initiated Amplification of Nucleic acids (ICAN) method (registered trademark), a Transcription-Reverse Transcription Concerted (TRC) method, a Strand Displacement Amplification (SDA) method, a Transcription-Mediated Amplification (TMA) method, and a Rolling Circle Amplification (RCA) method. Furthermore, the “nucleic acid amplification reaction” widely includes a nucleic acid amplification reaction which is performed under a varying temperature or isothermal condition so as to amplify nucleic acids. In addition, these nucleic acid amplification reactions also include a reaction, such as a real-time PCR method, in which quantification of the amplified nucleic acids is carried out.

A chamfered portion 116a is provided at one end of one side of the microchip 11a, and an angular portion 116b is provided at the other end thereof. Furthermore, notches 117 and 117 are provided on both lateral sides which are continuous to both ends of the one side of the microchip 11a. When the microchip 11a is inserted into the case 12 described below, the chamfered portion 116a functions as a structure for regulating an insertion direction of the microchip 11a into the case 12. Meanwhile, when the microchip 11a is received in the case 12 described below, the notches 117 and 117 function as a locking structure (a locking portion) for restricting movement of the microchip 11a in the case 12.

The microchip 11a is constituted to have a plurality of substrate layers. The microchip 11a has a configuration in which a substrate layer 111b on which the introduction portion 112, the well 113 and the passage 114 are formed, and a substrate layer 111c is stacked up on a substrate layer 111a, as shown in FIG. 2B, for example. Examples of material forming the substrate layers 111a, 111b, or 111c includes glass, plastic, metal, ceramic, and the like.

The substrate layer 111a which seals a space (the well 113, for example) in the microchip 11a is exposed at side end surfaces 115a and 115b of the microchip 11a. It is preferable that the substrate layer 111a which seals a space, such as the well 113, be formed of material having elasticity so as to enable piercing of the hollow needle 21 which is provided in the jig 2a described below. Examples of the material having elasticity include a silicone-based elastomer, an acrylic elastomer, a urethane-based elastomer, a fluorinated elastomer, a styrene-based elastomer, an epoxy-based elastomer, natural rubber, and the like. Particularly, it is preferable that the microchip 11a be constituted to have a plurality of the substrate layers 111a, 111b, and 111c which include a polydimethylsiloxane (PDMS), that is, the silicone-based elastomer, layer, such that the hollow needle 21 pierces the polydimethylsiloxane layer (the substrate layer 111a) when the liquid is injected into the microchip 11a by the liquid injection jig set A1 described below.

The substrate layer 111a into which the hollow needle 21 pierces is formed of material having elasticity. Thus, when the hollow needle 21 is pulled from the substrate layer 111a, subsequently to the piercing, a pierced portion is spontaneously sealed owing to a self-sealing property of the substrate layer 111a. In the present disclosure, the spontaneous sealing of the needle-pierced portion by elastic deformation of the substrate layer 111a is defined as the “self-sealing property” of the substrate layer 111a.

It is possible to form the introduction portion 112 or the like on the substrate layer 111b in an existing manner, such as wet etching or dry etching of a glass-based substrate layer, and nano imprint molding, injection molding, or cutting of a plastic-based substrate layer. The introduction portion 112, the well 113, and the passage 114 may be formed on the substrate layer 111b. Alternatively, parts of the introduction portion 112, the well 113, and the passage 114 are formed on the substrate layer 111b and the rest of the parts thereof are formed on the substrate layer 111a. These substrate layers 111a, 111b, and 111c are bonded to each other in an existing manner, such as thermal fusion bonding, bonding using adhesive, anodic bonding, bonding using an adhesive sheet, plasma activation bonding, or ultrasonic bonding.

When the substrate layers 111a, 111b, and 111c are bonded to each other, bonding of the substrate layer 111a to the substrate layer 111b is performed under a negative pressure condition ( 1/100 atm., for example) relative to atmospheric pressure. Thus, each space of the introduction portion 112, the well 113 and the passage 114 can be under a negative pressure condition relative to atmospheric pressure. The introduction portion 112 or the like is under the negative pressure condition, and thus it is easy to perform introduction of the liquid into the microchip 11a, which is carried out using the liquid injection jig set A1 described below.

To maintain the negative pressure condition of the space, such as the introduction portion 112, in the microchip 11a, it is preferable that the substrate layers 111b and 111c constituting outer surfaces of the microchip 11a be formed of a gas-impermeable material. Examples of the gas-impermeable material include glass, plastic, metal, ceramic, and the like. Examples of the plastic include polymethyl methacrylate: acrylic resin (PMMA), polycarbonate (PC), polystyrene (PS), polypropylene (PP), polyethylene (PE), polyethylene terephthalate (PET), diethylene glycol bis allyl carbonate, styrene-acrylonitrile copolymer (SAN resin), MMA-styrene copolymer (MS resin), poly(4-methylpentene-1) (TPX), polyolefin, (siloxanyl methacrylate monomer)-MMA copolymer (SiMA), SiMA-fluorine-containing monomer copolymer, silicone macromer(A)-HFBuMA (heptafluorobutyl methacrylate)-MMA ternary copolymer, di-substituted polyacetylene polymer, and the like.

Furthermore, in a case where the analyte held in the well 113 is analyzed in an optical manner, it is preferable that material with optical transparency, low autofluorescence, and small wave length dispersion allowing low optical error be selected as the material forming the substrate layers 111a, 111b, and 111c.

### (1-2) Case

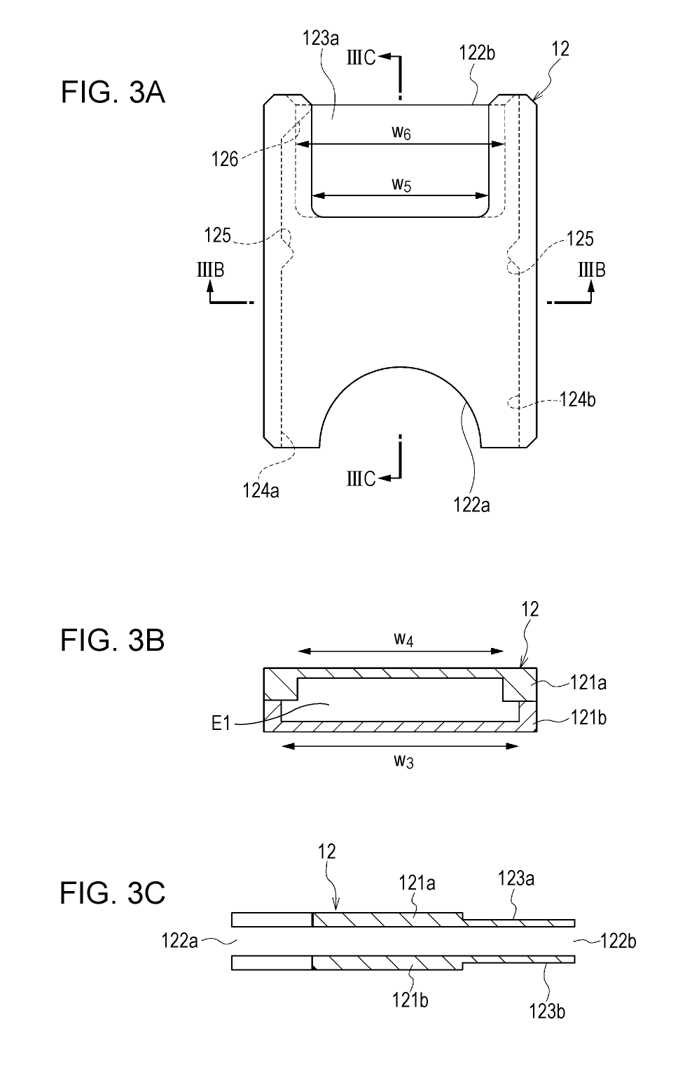

FIGS. 3A to 3C are schematic views of the case 12. FIG. 3A is a top view of the case 12, FIG. 3B is a cross-sectional view of the case 12 taken along line IIIB-IIIB indicated by arrows in FIG. 3A, and FIG. 3C is a cross-sectional view of the case 12 taken along line IIIC-IIIC indicated by arrows in FIG. 3A.

As shown in FIG. 3A, the case 12 has a first opening 122a into which the microchip 11a can be inserted, and a second opening 122b through which one side end surface 115a of the microchip 11a received in the case 12 is exposed to the outside. In addition, two parts of a case upper portion 121a and a case lower portion 121b constitute the case 12, and a space E₁ for receiving the microchip 11a described above is provided between the case upper portion 121a and the case lower portion 121b, as shown in FIG. 3B.

First convexes 125 and 125 are respectively provided on inside wall surfaces 124a and 124b of the case 12 so as to protrude toward the space E₁. These first convexes 125 and 125 are provided at positions where the notches 117 and 117 provided on the microchip 11a can be respectively locked into the first convexes 125 and 125 when the microchip 11a described above is received in the space E₁ (see FIG. 2A). As a result, the first convexes 125 and 125 and the notches 117 and 117 function as a locking structure (a locking portion) for restricting movement of the microchip 11a received in the case 12.

Furthermore, in one inside wall surface 124a out of the inside wall surfaces 124a and 124b which constitute an inner side of the case 12, a second convex 126 is provided in the vicinity of the second opening 122b. Along with the chamfered portion 116a formed on the microchip 11a described above, this second convex 126 functions as a structure for regulating an insertion direction of the microchip 11a into the case 12.

In a case where the microchip 11a is received in an inner space (the space E₁) of the case 12, when the microchip 11a is inserted into the space E₁ in a state where the chamfered portion 116a of the microchip 11a is positioned to be directed to the side of the inside wall surface 124a provided with the second convex 126, the microchip 11a is inserted into a predetermined position in the case 12 without being hindered by the second convex 126. In contrast, when the microchip 11a is inserted into the space E₁ in a state where the chamfered portion 116a is positioned to be directed to the side of the other inside wall surface 124b, the angular portion 116b of the microchip 11a bumps against the second convex 126, and thus it is difficult to insert the microchip 11a into the predetermined position. In this manner, the insertion direction of the microchip 11a into the case 12 is restricted, and thus the microchip 11a is prevented from being erroneously received in the case 12 from an opposite direction by a user.

The structure for regulating the insertion direction of the microchip 11a into the case 12 includes shape differences of the substrate layers 111a, 111b, and 111c, which constitute the microchip 11a, in addition to the chamfered portion 116a described above. In this case, a width w₁ of the substrate layer 111b is set to be greater than a width w₂ of the substrate layer 111a or 111c, for example (see FIG. 2C). In addition, the space in the case 12 is formed such that widths w₃ and w₄ of the case lower portion 121b and the case upper portion 121a are set to be different from each other, corresponding with widths w₁ and w₂ of the respective substrate layers (see FIG. 3B). As a result, it is difficult to insert the substrate layer 111b side into the case upper portion 121a side, and the insertion of the microchip 11a into the case 12 is allowed only in a direction where the substrate layer 111b side is positioned to be directed to the case lower portion 121b side.

The structure in the chip receiver 1a, which regulates the insertion direction of the microchip 11a into the case 12, is not limited to the structure described above, and any structure can be allowable as long as it can regulate the insertion direction of the microchip 11a into the case 12.

A first recess 123a and a second recess 123b are provided on an outer surface of the case 12, as shown in FIG. 3C. The first recess 123a and the second recess 123b are fitted to a plate portion of the jig 2a described below. In addition, a width w₅ of the first recess 123a and a width w₆ of the second recess 123b are set to be different from each other (see FIG. 3A). These first recess 123a and second recess 123b are formed to have different widths, and thus the first recess 123a and the second recess 123b function as structures for regulating a fitting direction of the chip receiver 1a to the jig 2a when the chip receiver 1a described below is fitted to the jig 2a.

Examples of the material forming case 12 include glass, plastic, metal, ceramic, and the like. Examples of the plastic include polymethyl methacrylate: acrylic resin (PMMA), polycarbonate (PC), polystyrene (PS), polypropylene (PP), polyethylene (PE), polyethylene terephthalate (PET), and the like. In addition, the case upper portion 121a and the case lower portion 121b may be formed of the same material or may be formed of different material.

### (2) Jig

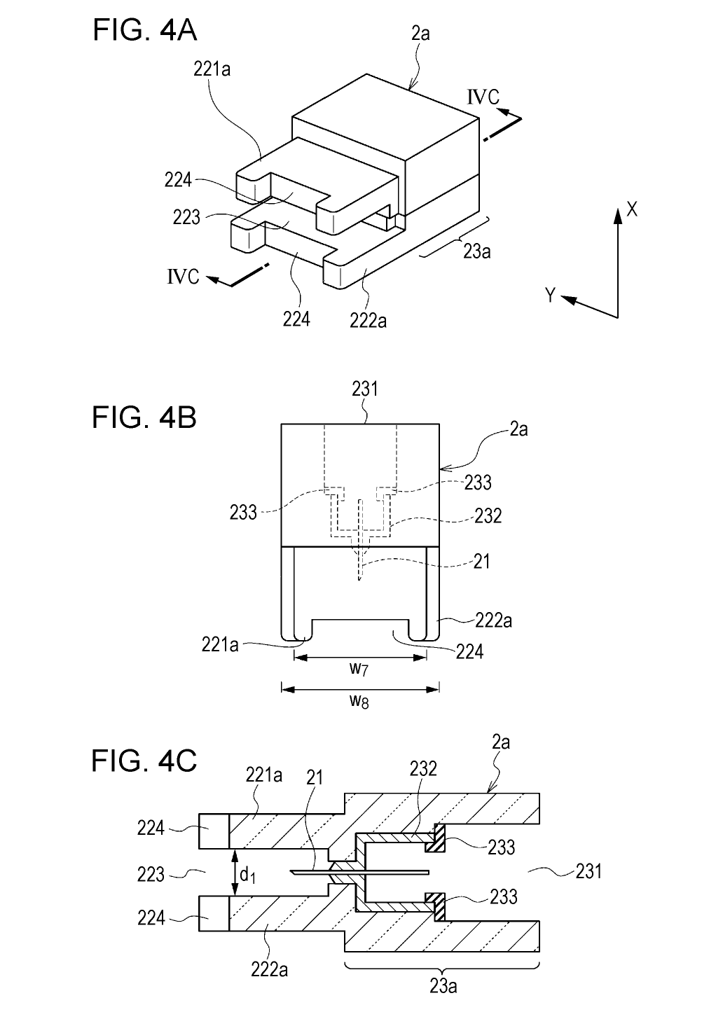

FIGS. 4A to 4C are schematic views of the jig 2a. FIG. 4A is a perspective view of the jig 2a when viewed from a plate portion (a first plate portion 221a and a second plate portion 222a) side, FIG. 4B is a top view of the jig 2a, and FIG. 4C is a cross-sectional view of the jig 2a taken along line IVC-IVC indicated by arrows in FIG. 4A. The jig 2a is configured to be fittable to the chip receiver 1a described above and has the hollow needle 21 that can pierce the side end surface 115a of the microchip 11a. Details of each configuration of the jig 2a will be described with reference to FIGS. 4A to 4C.

### (2-1) Plate Portion

The jig 2a includes a structure enabling pinching of the chip receiver 1a. Two opposite plate portions (the first plate portion 221a and the second plate portion 222a) are provided in the jig 2a, which is provided in the liquid injection jig set A1 according to the first embodiment of the present disclosure, as shown in FIG. 4A, for example. Thus, the chip receiver 1a is fittable to a gap 223 between the first plate portion 221a and the second plate portion 222a.

The first plate portion 221a and the second plate portion 222a function as a structure for allowing the chip receiver 1a to be positioned in and fitted to the jig 2a. In the jig 2a, a length d₁ of the gap 223 between the first plate portion 221a and the second plate portion 222a is substantially the same as the thickness of the chip receiver 1a (the case 12). Thus, the chip receiver 1a is positioned at a predetermined position by means of the two plate portions 221a and 222a. In the liquid injection jig set A1 according to the first embodiment of the present disclosure, the first plate portion 221 side is set to an upward direction and the second plate portion 222a side is set to a downward direction. Furthermore, for reasons of convenience, positioning with respect to the gap 223 between the first plate portion 221a and the second plate portion 222a is defined as positioning in an up-down direction (see arrow X shown in FIG. 4A).

In addition, the first plate portion 221a and the second plate portion 222a also function as a structure for allowing the chip receiver 1a to be positioned and fitted not only in the up-down direction but also in a right-left direction. Out of two directions perpendicular to a longitudinal direction of the hollow needle 21, a direction perpendicular to the up-down direction described above is defined as the right-left direction, for reasons of convenience (see arrow Y shown in FIG. 4A).

A width w₇ of the first plate portion 221a is substantially the same as the width w₅ of the first recess 123a of the case 12 described above and a width w₈ of the second plate portion 222a is substantially the same as the width w₆ of the second recess 123b of the case 12, as shown in FIG. 4B. Thus, when the chip receiver 1a is fitted to the jig 2a, the chip receiver 1a is positioned in and fitted to the jig 2a in the right-left direction.

Furthermore, a configuration of the chip receiver 1a and the jig 2a, which allows the chip receiver 1a to be positioned in and fitted to the jig 2a, is not limited to the configuration described above. Any configuration can be adopted as long as it allows the chip receiver 1a to be positioned in and fitted to the jig 2a, and a structure is not particularly limited.

In addition, the width w₅ of the first recess 123a and the width w₆ of the second recess 123b of the case 12 are set to be different from each other. Also, the width w₇ of the first plate portion 221a is substantially the same as the width w₅ of the first recess 123a and the width w₈ of the second plate portion 222a is substantially the same as the width w₆ of the second recess 123b. Thus, in a case where the chip receiver 1a is fitted to the jig 2a, an outer surface of the microchip 11a, which allows the fixation of the first plate portion 221a, is limited to the first recess 123a. In contrast, an outer surface of the microchip 11a, which allows the fixation of the second plate portion 222a, is limited to the second recess 123b. When these combinations are switched, the chip receiver 1a is not fitted to the predetermined position of the jig 2a. In such a manner, the first plate portion 221a and the second plate portion 222a which are provided on the jig 2a, and the first recess 123a and the second recess 123b which are provided on the chip receiver 1a also function as a structure for regulating a fitting direction of the chip receiver 1a to the jig 2a.

Furthermore, the structures in the chip receiver 1a and the jig 2a, which regulate the fitting direction of the chip receiver 1a to the jig 2a, are not limited to the structures described above, and the structures are not particularly limited as long as they can regulate the fitting direction of the chip receiver 1a to the jig 2a.

In addition, notches 224 and 224 are respectively provided in the first plate portion 221a and the second plate portion 222a (see FIGS. 4A and 4B). Thus, even in a case where the chip receiver 1a is fitted to the jig 2a in a manner such that the chip receiver 1a is pinched in the gap 223 of the jig 2a, the first plate portion 221a and the second plate portion 222a prevent a part of the chip receiver 1a, which corresponds to the well 113, from being covered. Thus, a user can visually confirm the well 113 formed on the microchip 11a (the reaction field) (see FIG. 1B).

Configurations which are applied to the first plate portion 221a and the second plate portion 222a to enable a user's visual confirmation of the well 113 are not limited to the notches 224 and 224. It may be configured in a way that a part of the first plate portion 221a or the second plate portion 222a is formed of material having optical transparency, and thus the well 113 can be visually confirmed through the material having optical transparency, for example. Furthermore, as long as the well 113 formed on the microchip 11a can be visually confirmed from at least one of the first plate portion 221a and the second plate portion 222a, it is sufficient to enable a user's visual confirmation of the well. In addition, to enable a user's visual confirmation of the well 113, it is also necessary for a part of the case 12, which corresponds to the well 113, to be formed of a material having optical transparency.

Examples of the first plate portion 221a and the second plate portion 222a includes glass, plastic, metal, ceramic, and the like. Examples of the plastic include polymethyl methacrylate: acrylic resin (PMMA), polycarbonate (PC), polystyrene (PS), polypropylene (PP), polyethylene (PE), polyethylene terephthalate (PET), and the like. In addition, the first plate portion 221a and the second plate portion 222a may be formed of the same material or may be formed of different material.

### (2-2) Hollow Needle

The hollow needle 21 is provided in the gap 223 between the first plate portion 221a and the second plate portion 222a, as shown in FIG. 4C. In a connection portion 23a described below, the hollow needle 21 is fixed to the jig 2a by a fixing member 232 so as to be connectable to a container B1 capable of containing liquid.

### (2-3) Connection Portion

The jig 2a is provided with the connection portion 23a for connecting the container containing liquid to the jig 2a, and the connection portion 23a has an opening 231. In addition, a packing 233 is provided in the connection portion 23a to ensure tight contact between the container and the opening 231. Furthermore, an O-ring may constitute the packing 233, for example.

### 2. Injection of Liquid by Liquid Injection Jig Set According to First Embodiment of Present Disclosure

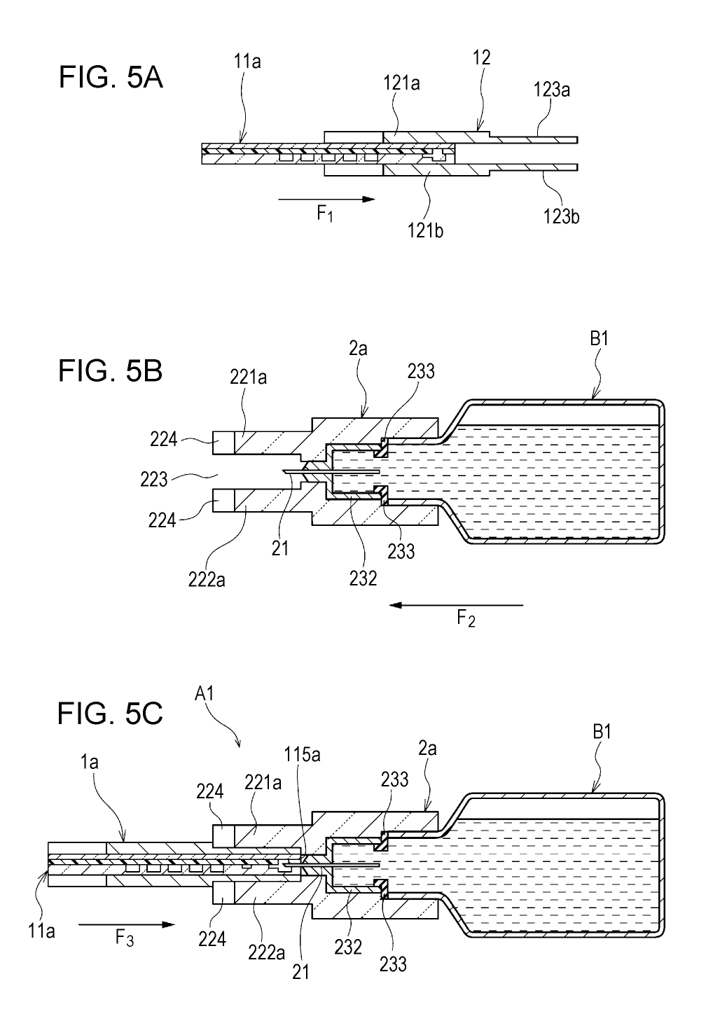

Details of injection of liquid into the microchip 11a using liquid injection jig set A1 will be described with reference to FIGS. 5A to 5C.

When the injection of liquid into the microchip 11a is conducted using liquid injection jig set A1, the microchip 11a is moved in a direction indicated by arrow F₁ and inserted into the case 12, as shown in FIG. 5A. In other words, the chip receiver 1a in which the side end surface 115a is exposed with the microchip 11a inserted into the case 12 is formed.

Meanwhile, in the jig 2a, the container B1 containing the liquid is pressed in a direction indicated by arrow F₂ and thus the container B1 is connected to the opening 231 provided in the connection portion 23a, and thus the container B1 is connected to the jig 2a, as shown in FIGS. 5A to 5C. Subsequently, the container B1 is in communication with the hollow needle 21 provided in the jig 2a. As a result, the liquid in the container B1 reaches the tip of the hollow needle 21 by the capillary action.

When the liquid reaches the hollow needle 21, the chip receiver 1a is fitted to the gap 223 between the first plate portion 221a and the second plate portion 222a, which is provided in the jig 2a (see arrow F₃ shown in FIG. 5C). When the chip receiver 1a is fitted to the gap 223, the hollow needle 21 provided between the first plate portion 221a and the second plate portion 222a perforates the side end surface 115a of the microchip 11a, which is exposed from the case 12. The tip of the hollow needle 21 perforates the substrate layer 111a and reaches the introduction portion 112, as shown in FIG. 5C.

In this case, if the inside of the microchip 11a is sealed so as to be in a negative pressure state relative to atmospheric pressure, the liquid is subjected to negative pressure suction into the microchip 11a after the hollow needle 21 pierces the microchip 11a.

In the liquid injection jig set A1 according to the first embodiment of the present disclosure, it is easy to perform, by means of the hollow needle 21, injection of the liquid into the well 113 or the like, which is provided in the microchip 11a. In addition, the liquid injection jig set A1 according to the first embodiment is provided with the structure for positioning the chip receiver 1a with respect to the jig 2a, and thus the side end surface 115a of the microchip 11a can be pierced, by the hollow needle 21, at a predetermined position. Therefore, the tip of the hollow needle 21 can be certainly positioned in the introduction portion 112 formed in the microchip 11a.

Furthermore, in the liquid injection jig set A1, the hollow needle 21 pierces from the side end surface 115a of the microchip 11a. Thus, by changing the position at which the introduction portion 112 is provided, it is possible to increase the thickness of the substrate layer 111a into which the hollow needle 21 pierces, without increasing the thickness of the microchip 11a. Thus, when material having elasticity, such as PDMS, is used for the substrate layer 111a, it is possible to enhance the self-sealing property of the portion pierced by the hollow needle 21.

Furthermore, in the liquid injection jig set A1, the hollow needle 21 is provided in the gap 223 between the two plate portions 221a and 222a which are provided in the jig 2a, and thus the hollow needle 21 is prevented from protruding from the jig 2a. Therefore, it is possible to prevent an accident that a user's hand or the like accidentally gets pricked by the hollow needle 21.

### 3. Configuration of Liquid Injection Jig Set According to Modification Embodiment of First Embodiment

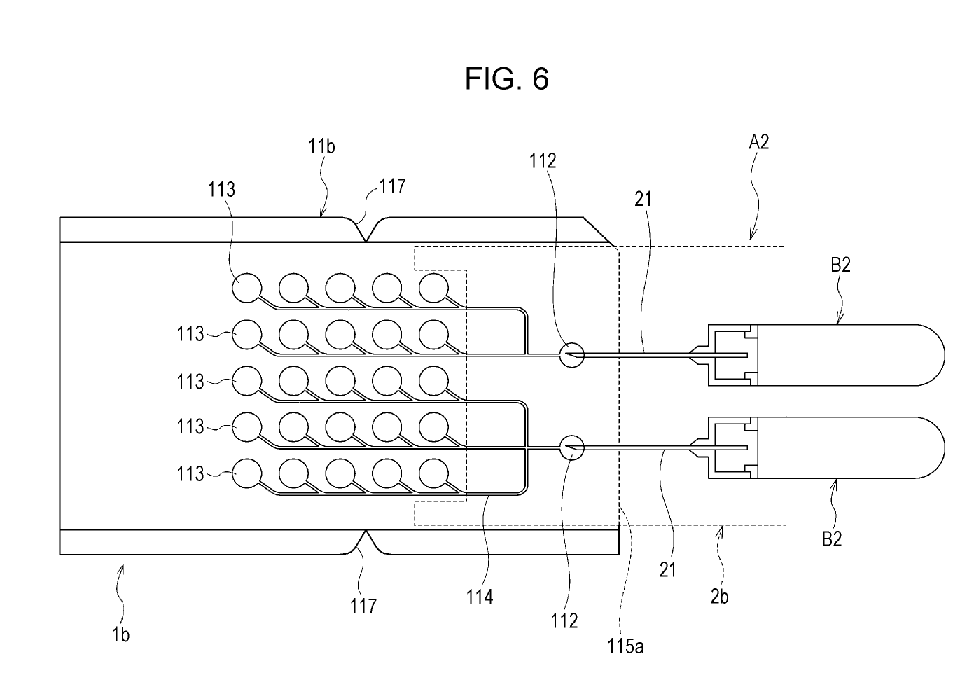

FIG. 6 is a schematic top view of the liquid injection jig set A2 according to a modification embodiment of the first embodiment and shows a state where the hollow needle 21 provided in a jig 2b pierces the side end surface 115a of a microchip 11b which is received in a chip receiver 1b. Further, the case 12 in which the microchip 11b is received is not included in FIG. 6.

The configuration of the liquid injection jig set A2 according to the modification embodiment of the first embodiment is the same as that in the first embodiment, except for the microchip 11b and jig 2b. The same reference numerals are given to components which are the same as those in the first embodiment, and the descriptions thereof will not be repeated.

Two introduction portions 112 and 112 are provided on the microchip 11b, as shown in FIG. 6. In addition, two hollow needles 21 and 21 are provided in the jig 2b so as to correspond to the introduction portions 112 and 112. One end of each of the hollow needles 21 and 21 is in communication with containers B2 and B2, respectively, separated from each other.

In the liquid injection jig set A2, a plurality of liquids having different compositions can be injected into the microchip 11b at the same time. Thus, analyses of a plurality of samples can be conducted at the same time using one microchip 11b. The other effects of the liquid injection jig set A2 are the same as those of the first embodiment.

### 4. Configuration of Liquid Injection Jig Set According to Second Embodiment of Present Disclosure

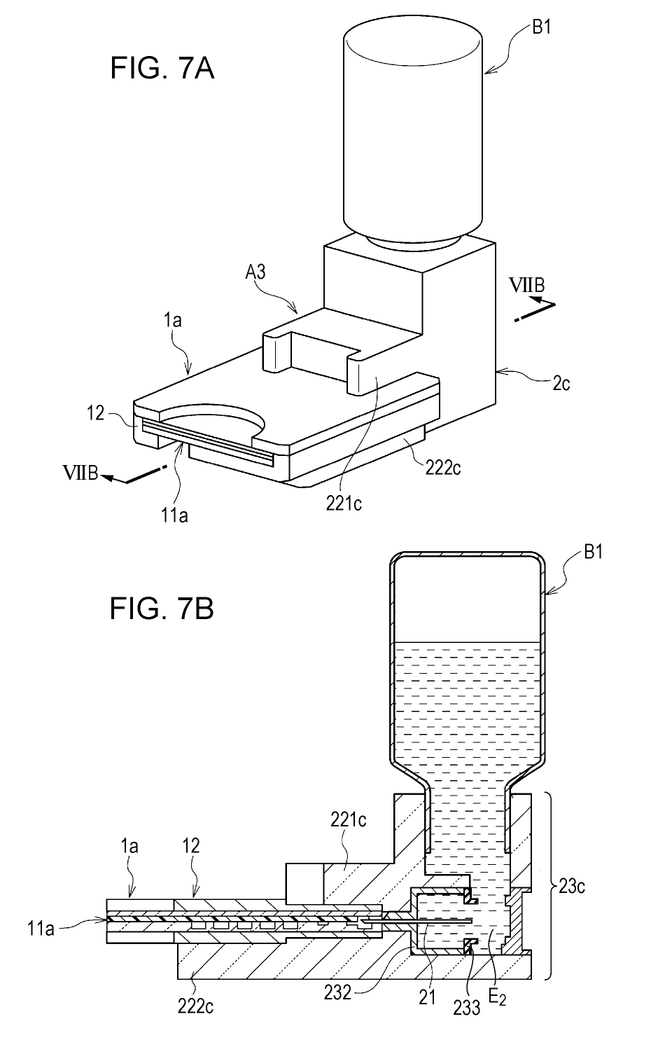

FIGS. 7A and 7B are schematic views of a liquid injection jig set A3 according to a second embodiment of the present disclosure. FIG. 7A is a perspective view of an external appearance of the liquid injection jig set A3 when viewed from a chip receiver 1a side, and FIG. 7B is a cross-sectional view of the liquid injection jig set A3 taken along line VIIB-VIIB indicated by arrows in FIG. 7A. Also, FIGS. 7A and 7B shows a state where the container B1 is connected to a jig 2c.

The configuration of the liquid injection jig set A3 according to the second embodiment is the same as that in the first embodiment, except the jig 2c. The same reference numerals are given to components which are the same as those in the first embodiment, and the descriptions thereof will not be repeated.

### (1) Connection Portion

In a connection portion 23c provided in the jig 2c, the hollow needle 21 does not directly communicate with the container B1 but is in contact with the container B1 via a space E2 in the connection portion 23c, as shown in FIG. 7B. In terms of the contact between the jig 2c and the container B1, any configuration can be applied to the liquid injection jig set A3 according to the present disclosure as long as the liquid can reach the tip of the hollow needle 21, as in the configuration described above. The configuration thereof is not particularly limited.

### (2) Plate Portion

A first plate portion 221c and a second plate portion 222c which are provided in the jig 2c are different in length and width, as shown in FIG. 7B. For reasons of convenience, the length of one side of the surface which constitutes a contact surface between the chip receiver 1a and the first plate portion 221c or the second plate portion 222c is defined as length in the description of the liquid injection jig set A3 according to the present disclosure. Further, the length of a side which is perpendicular to the side defining length is defined as width. Incidentally, in a case where the contact surface between the chip receiver 1a and the first plate portion 221c or the second plate portion 222c has an elliptical shape, the length and the width can be respectively substituted by a long side and short side of the ellipse.

In the liquid injection jig set A3 according to the second embodiment of the present disclosure, the space E2 is provided to allow the container B1 to communicate with the hollow needle 21. Thus, a contact direction of the container B1 to the jig 2c can be directed in a predetermined direction, as well. When the container B1 is brought into contact with the jig 2c in a state where an opening portion of the container B1 is directed downward, as shown in FIG. 7B, for example, air in the container B1 is collected in the upper part of the container B1. Thus, it is possible to easily prevent the air from being injected into the microchip 11a via the hollow needle 21.

In the liquid injection jig set A3 according to the second embodiment of the present disclosure, the first plate portion 221c and the second plate portion 222c are different in length and width. In a case where the jig 2c is put on a table or the like, the jig 2c is placed on the table with a higher stability because the length and the width of the second plate portion 222c which is brought into contact with the table are comparatively greater than those of the first plate portion 221c. In addition, improvement in placement stability of the jig 2c on the table is achieved even in a case where either one of the length or the width of the second plate portion 222c is comparatively greater than that of the first plate portion 221c.

Furthermore, in a case where the first plate portion 221c and the second plate portion 222c are different in length and width, it is possible to fit the chip receiver 1a to the gap 223 between the first plate portion 221c and the second plate portion 222c in such a manner that, first, the chip receiver 1a is brought into contact with one plate portion of which the length or the width are greater than those of the other plate portion and then the chip receiver 1a is slid thereon and fitted to the gap. Thus, it is easy to perform fitting of the chip receiver 1a. The other effects of the liquid injection jig set A3 are the same as those of the first embodiment.

The present disclosure can be configured as follows.

(1) A liquid injection jig set including a chip receiver in which a microchip having a plate shape is received in a case, and a jig that is configured to be fittable to the chip receiver includes a hollow needle that can pierce a side end surface of the microchip, which is exposed from the case, and introduces liquid into the microchip via the hollow needle.

(2) The liquid injection jig set according to (1) described above, in which the chip receiver is positioned to and fitted to the jig.

(3) The liquid injection jig set according to (2) described above, in which the chip receiver is positioned in a right-left direction and an up-down direction.

(4) The liquid injection jig set according to any one of (1) to (3) described above further including a structure that regulates a fitting direction of the chip receiver to the jig.

(5) The liquid injection jig set according to any one of (2) to (4) described above, in which the jig has a structure enabling pinching of the chip receiver.

(6) The liquid injection jig set according to (5) described above, in which the jig includes two plate portions opposite to each other, and the chip receiver is fitted to a gap between the two plate portions.

(7) The liquid injection jig set according to (6) described above, in which the respective plate portions are different in length and/or width.

(8) The liquid injection jig set according to (6) or (7) described above, in which a reaction field formed in the microchip can be visually confirmed through at least either one of the two plate portions, in a state where the chip receiver is fitted to the jig.

(9) The liquid injection jig set according to any one of (1) to (8) described above, in which the liquid is subjected to negative pressure suction into the microchip when the hollow needle pierces the microchip.

(10) The liquid injection jig set according to any one of (1) to (9) described above, in which the microchip is configured to have a substrate layer including a polydimethylsiloxane layer and the hollow needle pierces the polydimethylsiloxane layer.

(11) A chip receiver including a microchip having a plate shape, and a case capable of receiving the microchip, in which the case is configured to be fittable to a jig that has a hollow needle that can pierce a side end surface of the microchip and introduces liquid into the microchip via the hollow needle and the side end surface of the microchip is exposed in a state where the microchip is inserted in the case.

(12) The chip receiver according to (11) described above further includes a structure that regulates an insertion direction of the microchip into the case.

(13) The chip receiver according to (11) or (12) described above, in which the chip receiver includes a locking portion that restricts movement of the microchip received in the case.

It should be understood by those skilled in the art that various modifications, combinations, sub-combinations and alterations may occur depending on design requirements and other factors insofar as they are within the scope of the appended claims or the equivalents thereof.

What is claimed is:

1. A liquid injection jig set comprising:
a chip receiver in which a microchip having a plate shape is received in a case; and
a jig that is configured to be fittable to the chip receiver includes a hollow needle that can pierce a side end surface of the microchip, which is exposed from the case, and introduces liquid into the microchip via the hollow needle.

2. The liquid injection jig set according to claim 1,
wherein the chip receiver is positioned to and fitted to the jig.

3. The liquid injection jig set according to claim 2,
wherein the chip receiver is positioned in a right-left direction and an up-down direction.

4. The liquid injection jig set according to claim 1, further comprising:
a structure that regulates a fitting direction of the chip receiver to the jig.

5. The liquid injection jig set according to claim 2,
wherein the jig has a structure enabling pinching of the chip receiver.

6. The liquid injection jig set according to claim 5,
wherein the jig includes two plate portions opposite to each other, and the chip receiver is fitted to a gap between the two plate portions.

7. The liquid injection jig set according to claim 6,
wherein the respective plate portions are different in length and/or width.

8. The liquid injection jig set according to claim 7,
wherein a reaction field formed in the microchip can be visually confirmed through at least either one of the two plate portions, in a state where the chip receiver is fitted to the jig.

9. The liquid injection jig set according to claim 8,
wherein the liquid is subjected to negative pressure suction into the microchip when the hollow needle pierces the microchip.

10. The liquid injection jig set according to claim 9,
wherein the microchip is configured to have a substrate layer including a polydimethylsiloxane layer, and
wherein the hollow needle pierces the polydimethylsiloxane layer.

11. A chip receiver comprising:
a microchip having a plate shape; and
a case capable of receiving the microchip,
wherein the case is configured to be fittable to a jig that has a hollow needle that can pierce a side end surface of the microchip and introduces liquid into the microchip via the hollow needle, and
wherein the side end surface of the microchip is exposed in a state where the microchip is inserted in the case.

12. The chip receiver according to claim 11, further comprising:
a structure that regulates an insertion direction of the microchip into the case.

13. The chip receiver according to claim 12,
wherein the chip receiver includes a locking portion that restricts movement of the microchip received in the case.

14. A jig comprising:
a structure which enables fitting of a chip receiver in which a microchip having a plate shape is received in a case; and
a hollow needle that can pierce a side end surface of the microchip, which is exposed from the chip receiver,
wherein liquid is introduced into the microchip via the hollow needle.
